SEQUENCE LISTING (3 sequences)

SEQ ID NO: 1 - DNA, 3591 bp, Zebrafish
gaattcactaatttgaatttaaatgcattatctttctattcctaaagaccttgggtgaccaaaatcttattttaataaataaaactgtttattaaaacttttttgtttcaaagaaccatatgtatagtgaaatttataaaaatatcaatttttaaaaagctggtgtactcatttatgttatgaactctaaaaccatatactgactgcaagtgatgatgtatagagtgatgtttacgagtaaacatatttagttgtatacatcctactgagcacattttgatgtatgaaataacattacaagctttacccaaattaagccattttaaaacactgccaattgaaaatacaaatcctggaaaaaatcgtctttagcgcagtcatttgagccatcctaatccgttacctcagaccataataagaagggataacactagctgtagcaatggaacacatctgtttcacacaatcatatctcctgcgccggtgctaagcagattcagcgtgatcataacatgctttccactcataaatgtaaatttacaatttgcacatgtaaaacagacacttttgagatattggataaaaaaacaagagtatattgcttagtttcatccaccagtcatccccacagcgtttggaaggccataaaaagtgtctaaaatcaatgatcattgaaagagcacaagagagactcttacgctgtaatgccactggggacaaaagtgacagtctcttaatgggctcttctggaggggctcctgaacattaaaaattatcagcgaaattaccgaaagagcttcaagcaactggcatgcttgatcctctgcgtcggggcggtgaataggtgcttcagatgccctcttacccacgggctggattcagctgccccgctaccagcggagaccccctaatgagcctctgcaattaagtttattcatgttaagtgtgaacggggtgcgtgcggaactgtgggcagctaacagacctgggttctttgtgccacaagtgctgcctttattcggctcacaaagcagaaaacaacacccgcacctataatggcgccctcggctgggtctaagaaacgtggcgagttgacagagcagagtgggcggggttaagacagactgacagcgggacccatctccatcctcttattaacgcttaacgagtgccttcctcatgcaatattcatcgccactaatatcatccaagctctgagctgagctggccacttatgtaaggcaattatgtaaaatatcagacagggcccacactcagaatctgactggggtagagacgcgggacgagaaccgagagcaagaactgaaagtgaaagtgaccactaaagggaggagaggacagaggggcaggatgtgtcaagattaccagagaacacttggccagaaatgcgcaaccattggagctctccggattacccaaaggttaacgagtttgaacgcctctgcccactcgccatctctgatggtttcccaagaactcctcaagcaaaatatatataattgtgtgtattatgcacagacacgagaaaatgctgtttttctgatctgcattacagcacatttgcccgccaacgacaataccacccactcggtacctcgctgactcctgatgcctgatacctgcgcggtgactgtctacaatctgcataatcaagagaagttgtgttgaagacgagcgccacacaaccgtttccacaaggtcacccaaggccggtgcagatgtaggtgaggtctccataaacagactgaaataaacacatcctccgctgggaacaacaaccccctcacgcctcatgcattccataagcctacatgcatctcttccaacttatggagactcgcacctaccaacatccgcacaacaaagatatacagagcgcgctccctcaggtcaaggcctgtgggggtctgtgcagaaataggtcatttgtcacacatcaagtcctggggcaggagatgcattatagatgagaccaaacagcctgtctcggtgagctctacccactccctgagactagaaatgggggaagggagcttgagataacaaccgctgcaatcactgtgtcgatgttaatatcagcaccaaccgggaacaataaggagatggatgcattcatgttcacatcttaccagtcaagtatcatcgaaccggcttgataaccacacctcgtgtaatagctgagcagatagttgtcattttaaagcgttggcctttgtcgattatgtaatgcgcacattcaacacatggtaatatagaaacggttatgtcgaggttgttttgtccagagatgaccttcacacagttacagccgctctgcatccacacaaatggaggacttaatcgtggactgcattcttagaaatgatctacaaagacaaataatgtgaaatcaagaaaggacaaaatttaagtaaggggatgagggagagagagaacgaggggcaaggagaaagcatggctcctgtctttttctgcacccatctgttcggagtgcaggtggagctctattcactcagctctgcatgtgtgtttggggggggcaggaagaaagggagggcaaaaggaagagtggagagatggtgggggctggagggatggggggttctcggtgatctctcctgaaggggataatgggagagcagcgctttgcaatggctgccatgtagtaccctccctgcacaattagccaatcagcagcaactctgccagccagaaggacacataaagaagaacattgcagcagaggcacagaaggagcctgcgaggagctgggaaatacacacacaacagcagaaccacaacaccctcccctggacacaccctactggggatcactgcttttctttttttctgaaccatcgcccacgccacacggagagaaatctctctctcatcatcatcctgaagaaaacccccttatcctcattttcacactgctgaggaaaacctacaatcgcacgggctgagatttcctggcgaagactgtcctttttcctttttctttttttttcttttcctttggaaactgacatttgcatttctccattccaagccacggcgtaataatatctgcaatccagcctgaagactgcaaatcgaaggactagatcatatcatctttgtacgtcaagaatggttactgtacgtataacctttctttcttttgctctgaccaatatgaaacactaaaatcgattcgagcagcctctcagcatcaattacagtgcgtgaaaaacattcaaatagaggcagcaaatattacatgtgaaaatacaggctggctaaatccaagctaatttagaaatgtggtcaaaacgcatactggcacgtctaatcgcggattcagtaaacaagattaacgattagcccagtgtataagtcatatgatacagcatgcgcgagagcatcgctacacccgagctggcttcattttcggaggaaaatcaaaacattgctttctcctgccgtgcgaaccattcgtcataaaccgtaatacgcaacatacatttattactacatccgttaattagcgataattagccgttattaacaaagagcgctgaggaattc

SEQ ID NO: 2 - DNA, 24 bp, Artificial
  primer
cctcagcagtgtgaaaatgaggat

SEQ ID NO: 3 - DNA, 24 bp, Artificial
  primer
tagcccatcagcagcaactctgcc

(19) United States
(12) Patent Application Publication
(10) Pub. No.: US 2004/0093630 A1
(43) Pub. Date: May 13, 2004

(54) Zebrafish huc promoter capable of directing neuron-specific expression of structural genes, transgenic animal having huc promoter and its generation, and method for screening neuronal mutant animals using the transgenic animal

(76) Inventors: Tae Lin Huh, Daegu (KR); Hael-Chul Park, Daegu (KR); Chul-Hee Kim, Kyungbuk (KR); Hyung-Seok Kim, Daegu (KR)

Correspondence Address: Joseph Hyosuk Kim, JHK Law, PO Box 1078, La Canada, CA, 91012-1078, US

(21) Appl. No.: 10/296,665

(22) PCT Filed: Jul. 24, 2001
(86) PCT No.: PCT/KR01/01259

(30) Foreign Application Priority Data
Aug. 28, 2000 (KR) 2000-50017

(51) Int. Cl. A01K 67/027; C07H 21/04; C12P 21/02; C12N 5/06

(52) U.S. Cl. 800/20; 435/69.1; 435/320.1; 435/325; 536/23.5

(57) ABSTRACT

The present invention relates to a zebrafish HuC promoter with its 5′-flanking region which directs the neuron-specific expression of structural genes, a transgenic animal that shows the neuron-specific expression of GFP (green fluorescence protein) under the regulation of the HuC promoter, and a method for screening neurogenesis mutants in zebrafish by use of the transgenic animal. The HuC promoter with its 5′-flanking region can be used in the study of the regulatory mechanism responsible for the differentiation of the nervous system. Additionally, the HuCP-GFP transgenic zebrafish enables the direct identification of neurogenesis and axonogenesis, as well as being a valuable tool for isolating and analyzing neurogenesis mutants in live zebrafish with ease.

### FIELD OF THE INVENTION

The present invention relates to a zebrafish HuC promoter that drives the neuron-specific expression of structural genes, and a transgenic animal having the HuC promoter and its generation. Also, the present invention is concerned with a method for screening neuronal mutants, using the transgenic animal.

### BACKGROUND OF THE INVENTION

In gene expression, transcriptional regulation is very important for rapid responses to external signals and establishment of development. Primary spatial and temporal regulation of gene expression is conducted at the transcription level, in which transcription regulatory proteins recognize specific DNA sequence regions near promoters to specifically control the synthesis of mRNA. To express a certain gene in a specific tissue and/or at a specific time, the promoter of the gene and neighboring regions to which transcription regulatory proteins bind are therefore momentous.

Besides transcription factors, factors that are involved in the regulation of biosynthesis of proteins from gene information include those that are related to the stability of mRNAs produced from genes and that serve to carry mRNAs to the cytosol, particularly, to designated locations within the cytosol. Not only do proteins that play certain roles in the regulation of gene expression have motifs which recognize specific sites of mRNAs, but also expression of their genes are tissue-specific or time-specific according to development stages (Burd and Dreyfuss, 1994).

Belonging to a family of vertebrate neuron-specific genes, HuC is known to be highly homologous to the Drosophila elav, a vital gene indispensable for the development and maintenance of the nervous system (Good, 1995; Kim et al., 1996). Although much needs to be done to elucidate its functions, vertebrate HuC protein was reported to be able to bind AU-rich 3′-untranslated regions (UTRs) of mRNAs for various transcription factors and cytokines and thus believed to play an important role in postmitotic neuronal differentiation and subsequent maintenance of the vertebrate nervous system (Levine et al., 1993; King et al., 1994; Liu et al., 1995; Ma et al., 1996b; Chen and Shyu, 1995).

Essential to the development and maintenance of the nervous system, the Drosophila elav protein is the first case of a RNA-binding protein which is expressed specifically in neuronal tissues. Drosophila elav was identified on the basis of its RNA-binding motif, which suggests that the elav protein might be related to neuronal RNA metabolism (Robinow et al., 1988).

Studies on elav proteins in the whole developmental process using antibodies have disclosed that the elav protein 1) is expressed during the early stage of neuronal differentiation, 2) appears throughout the central nervous system and peripheral nervous system during the progression of nervous system development, 3) is translocated into nuclei, and 4) is not found in neuroblasts nor glial cells (Robinow et al., 1988, 1991). These results lead to the inference that elav functions as a housekeeping gene required for the development and maintenance of neurons.

Due to its requirement in neurons from an early stage of differentiation, elav has been used as an early neuronal marker and examination of its expression has helped study cellular, molecular, and genetic interactions that control early neurogenesis in Drosophila (Campos et al., 1987; Robinow and White, 1988). HuC, a vertebrate homologue of elav, has been suggested as a useful tool in the study of early neurogenesis in zebrafish (Kim et al., 1996) as recent studies have emphasized similarities in the mechanisms that control early neurogenesis in Drosophila and vertebrates, particularly in zebrafish and Xenopus embryos.

In zebrafish, early neurons are distributed in three longitudinal columns of the neural plate. Within these longitudinal columns only a subset of cells express HuC and differentiate into neurons. Neurogenin1 (ngn1), a basic helix-loop-helix (bHLH) transcription factor, is limitedly found only in the longitudinal domains where cells have the potential to become neurons, among the distributed columns. That is, the expression of neurogenin1 (ngn1) helps define the longitudinal proneuronal domains (Blader et al., 1997; Kim et al., 1997; Korzh et al., 1998). However, ngn1 drives the expression of the inhibitory ligand DeltaA, which interacts with its receptor, Notch, in neighboring cells whose activation, in turn, reduces the expression of ngn1 in these cells. As a consequence of this inhibitory feedback loop, only a subset of cells manage to maintain high levels of ngn1 and DeltaA expression (Appel and Eisen, 1998; Haddon et al., 1998)). Cells that do these feedback operations begin expressing another Delta homologue, DeltaB and genes like MyT1 and Zcoe2 that facilitate the stable adoption of a neuronal fate (Bellefroid et al, 1996; Bally-Cuif el al., 1998). These cells also begin to express neuroD, another bHLH transcription factor whose activity leads to expression of early markers of neuronal differentiation like HuC (Korzh et al., 1998). Neighboring cells, in which neuronal fate is suppressed by Notch activation, adopt alternate fates, or remain undifferentiated, giving rise to neurons later in development. When the function of the neurogenic genes like Notch and Delta is suppressed, loss of lateral inhibition leads to the overproduction of HuC-expressing cells (Appel and Eisen, 1998).

Zebrafish are now widely used in genetic screening to identify genes responsible for a range of early developmental events. They are particularly well suited to genetic analysis because large numbers of embryos can be easily obtained and raised to maturity within a relatively short period. Furthermore, the embryos are completely transparent during the first day of development (Chitins and Kuwada, 1990, Wilson et al., 1990).

Through large-scale mutagenesis screening, there have been already identified a number of mutants in which the early pattern of neurons is altered, for which the expression of HuC was used as an early neuronal marker. In this regard, in order to identify zebrafish mutants in which the distribution of HuC mRNA is altered, an approach was used where the embryos were screened for changes in the distribution of HuC-expressing cells by in situ hybridization. The success of this screening demonstrated the value of HuC as an early neuronal marker. However, RNA in situ hybridization suffers from the disadvantage of making it impossible to directly observe changes in the nervous system of live embryos because the chemicals used for the hybridization kill the embryos. Another problem with the screening method using RNA in situ hybridization is that a complex, time-consuming procedure such as mRNA synthesis, etc. is required. Accordingly, conventional screening methods using in situ hybridization cannot be applied for live embryos owing to their limitations in screening neurogenesis mutants in live embryos and analyzing alterations of neurogenesis therein. Therefore, there remains a need for an improved method that is able to directly identify and analyze alterations in early patterns of neurons of living embryos.

### SUMMARY OF THE INVENTION

Leading to the present invention, the intensive and thorough research on the early stages of differentiation of neurons resulted in the finding that 2.8 kb of the 5′-flanking sequence of a zebrafish HuC gene is sufficient to restrict GFP (green fluorescence protein) gene expression to neurons, in which the core promoter spans 251 base pairs and contains a CCAAT box and one SP1 sequence, while no TATA boxes are present near the transcription initiation site. It was also found that a putative MyT1 binding site and at least 17 E-box sequences are necessary to maintain the neuronal specificity of HuC expression. Sequential removal of the putative MyT1 binding site and 14 distal E boxes leads to a progressive expansion of GFP expression into muscle cells. Further removal of the three proximal E boxes eliminates neuronal and muscle specificity of GFP expression and leads to ubiquitous expression of GFP in the whole body. Using the HuC promoter, a stable zebrafish transgenic line (HuCP-GFP) can be established in which GFP is expressed specifically in neurons. By taking advantage of this stable zebrafish transgenic line, neurogenesis mutants in live zebrafish can be visibly identified with ease.

Therefore, it is an object of the present invention to provide a HuC promoter that drives the neuron-specific expression of structural genes.

It is another object of the present invention to provide a fused gene construct in which an exogenous GFP gene is expressed under the regulation of the HuC promoter.

It is a further object of the present invention to provide a transgenic animal which harbors the fused gene construct in its genome.

It is still a further object of the present invention to provide a method for generating the transgenic animal.

It is still another object of the present invention to provide a method for screening and analyzing neurogenesis mutants in live zebrafish embryos.

### BRIEF DESCRIPTION OF THE DRAWINGS

FIG. 1 shows fluorescence photographs which compare the expression of HuC (A) and DeltaB (B) mRNA in the neuronal plate at the 3-somite stage in dorsal views with anterior to the left. In this figure, ps stands for primary sensory neuron; pin for primary intermediate neuron; and pmn for primary motor neuron.

FIG. 2 is a base sequence showing the structure of the 5′-flanking region, including promoter, of the zebrafish HuC gene, in which various symbols or letters are used to denote special functions. The major transcription initiation site is presented as position +1 and marked by an arrow. The shaded letters mark the exon-1 and underlined lowercase letters denote the oligonucleotide sequence corresponding to the antisense oligonucleotide primer used for primer extension. Bold letters ATG stand for the translation start codon, MyT1, GATA-1 and SP1 sites are underlined. The canonical CBF/Ny-Y binding site (CCAAT-box) is double underlined and E-boxes are boxed.

FIG. 3 is an autoradiogram showing the determination of the transcription initiation site of HuC gene by primer extension.

FIG. 4 is a schematic diagram showing the structure of the zebrafish HuC promoter in embryos, along with their transient expression patterns of GFP in neurons, muscle cells and other tissues upon the introduction of deletion constructs.

FIG. 5 shows photographs taken of live, 48-hpf zebrafish embryos microinjected with ΔEco under a photo-field, which show the neuronal specificity of gene expression driven by the HuC promoter construct visualized through the transiently expressed GFP fluorescence

(A) generated by superimposing a bright-field image on a fluorescence image throughout the whole body with the dorsal part at the top and the anterior to the left;

(B) detected in the nervous system including the telencephalic cluster, retinal ganglion cells, medial longitudinal fasciculus, and dorsal longitudinal fasciculus;

(C) detected in the trigeminal ganglion neuron and Rohon-Beard neurons (arrows); and

(D) and (E) detected in the peripheral process of Rohon-Beard axons (arrow) and dorsal longitudinal fasciculus of spinal cord. Throughout the photographs, the dorsal part and the anterior part are located at the top and the left, respectively, and the abbreviation dlf stands for dorsal longitudinal fasciculus; ey for eye; mlf for medial longitudinal fasciculus; rb for Rohon-Beard neurons; rg for retinal ganglion; tc for telencephalic cluster; and tg for trigeminal ganglion.

FIG. 6 shows photographs taken of live, 48-hpf zebrafish embryos, which exhibit GFP expression patterns for functional analysis of deletion constructs,

(A) when ΔHind construct was microinjected into one-cell stage embryos;

(B) when ΔBst construct was microinjected into one-cell stage embryos;

(C) when ΔSac construct was microinjected into one-cell stage embryos; and

(D) when ΔSac construct was microinjected into four-cell stage embryos.

FIG. 7 shows photographs taken of live transgenic zebrafish embryos, which exhibit GFP fluorescence detected in

(A) the neurons of a 24-hpf heterozygotic transgenic embryo in a lateral view;

(B) the neurons of a 24-hpf homozygotic transgenic embryo in a lateral view;

(C) the cranial ganglia highlighted by asterisks and in ventral motor roots of the boxed area marked by arrows; and

(D) the spinal cord of a 60-hpf transgenic zebrafish embryo in a lateral view. In these figures, rb stands for Rohon-Beard cells, co for commissural neurons, and mo for primary motorneurons.

FIG. 8 shows photographs taken of the homozygotic transgenic zebrafish embryos, which exhibit temporal and spatial expression patterns of the HuCP-GFP fused gene construct,

(A) detected by whole mount in situ hybridization using a synthetic antisense RNA probe for GFP mRNA transcripts in a dorsal view of an 11-hpf embryo;

(B) detected by whole mount in situ hybridization using a synthetic antisense RNA probe for HuC mRNA transcripts in a dorsal view of an11-hpf embryo;

(C) through the expression of acetylated α-tubulin detected by whole mount immunostaining in a lateral view; and

(D) detected in the telencephalic cluster (tc), anterior commissure (ac), epiphysial cluster (ec), posterior commissure (pc), tract of posterior commissure (tpc), postoptic commissure (poc), and tract of the postoptic commissure (tpoc) of a 24 hpf embryo by anti-GFP antibodies in a lateral view;

(E) detected in the olfactory placodes in an anterior view;

(F) detected in medial longitudinal fasciculus (MLF) and its nucleus (nMLF) in a dorsal view;

(G) detected in the trigeminal ganglion (tg) and rhombomeres (v) in the hindbrain in a dorsal view of the hindbrain.

FIG. 9 is a photograph showing living mib mutant transgenic embryos visualized by GFP fluorescence, in which the neurogenic phenotype in 2-day-old HuCP-GFP^{+/-}/mib^{-/-} zebrafish embryo seen by GFP fluorescence with a Leica MZFLIII fluorescence stereomicroscope (right) is compared with a heterozygotic wild-type HuCP-GFP^{+/-} transgenic embryo (left).

### DETAILED DESCRIPTION OF THE INVENTION

In one aspect of the present invention, there is provided a HuC promoter governing the regulation of which structural genes are specifically expressed in neurons.

HuC, which is expressed from HuC, belongs to the Hu family of proteins which have RNA-recognition motifs and are a type of RNA-binding proteins which take part in RNA metabolism, such as rRNA production, translation initiation, structural RNA production, and transportation of RNA to the cytoplasm. Of the human Hu proteins identified thus far, HuD, HuC and He1-N1 are each found to have three RNA-recognition motifs and share a homology of as high as 86-90% with one another. Indispensable for the neuron-specific local expression and the development and maintenance of the nervous system, such proteins are believed to play an important role in neurogenesis and its control in vertebrates, like Drosophilia elav protein, when account is taken of the high homology between elav, and HuD, HuC and He1-N1.

In neurogenesis, clusters of cells must be separated and undergo mitosis to develop into differentiated cells, that is, neurons. In this development, the Hu protein may be a useful marker. In the case of zebrafish embryos, HuC is expressed at high levels during the whole neurogenesis process, beginning with the first expression in the proneuronal domains of the neural plate (Kim et al., 1996a). Hu proteins which show neuron-specific expression are complementary to other RNA-binding proteins which are encoded by murine musashi (Sakakibara et al., 1996). The murine musashi gene is expressed in neural stem cells. When the cells are differentiated to neurons, musashi ceases to be expressed, but the expression of Hu proteins starts. While the musashi gene is responsible for the control necessary for differentiation and the maintenance of mitotic cells, Hu genes function to control differentiation-relevant genes and maintain the differentiated cells. In consequence, musashi suppresses differentiation whereas Hu suppresses proliferation (Okano, 1995). It is inferred that Hu proteins associate with certain domains of RNA through their RNA-binding motifs to control their expression during neurogenesis.

Zebrafish is an important model that provides clues to understanding the early control of neurogenesis in vertebrates because it has a relatively simple nervous system and many genes responsible for a range of early developmental events have been identified. They are particularly well suited to genetic analysis by virtue of the fact that large numbers of embryos can be easily obtained and raised to maturity within a relatively short period. Furthermore the embryos are completely transparent during the first day of development at the time of which their nervous system is established, so it is easy to observe the developmental events.

Identification of the HuC promoter is prerequisite to investigate the mechanism in which the neuron-specific expression of HuC is controlled. In the present invention, the HuC promoter, which is extensively used as a useful tool in the study of early neurogenesis in zebrafish, was isolated and analyzed so as to study cellular, molecular, and genetic interactions that control early neurogenesis in vertebrates.

The HuC promoter provided by the present invention has a transcription start site which starts with G (see FIG. 2). The transcription start site mapped at G is consistent with the report that RNA polymerase II prefers to start at purines (Baker and Ziff, 1981). The presence of one CCAAT box (−64/−60), one GATA-1 (−241/−238) and one SP1 (−213/−208) site were revealed to be present in the immediate upstream region of the transcription start site, suggesting the possibility that the core promoter for HuC is located around this region. However, there is no obvious TATA box near the region 30-bp upstream of the transcription start site. The most striking feature of the 5′-flanking sequence of the HuC gene is the presence of 18 E-box sequences, which indicates that E-box-binding bHLH transcription factors (Murre et al., 1994) take an important part in the neuron-specific regulation of HuC gene expression. This is consistent with the previously suggested role of bHLH transcription factor like ngn1 in determination of neuronal fate. Additionally, one putative MyT1 binding site, which has also been reported to be essential for neuronal differentiation, was identified at nucleotide position −2687/−2680.

In another aspect of the present invention, there are provided a fused gene construct in which the HuC promoter and genes under the regulation of the HuC promoter are combined, and a transgenic animal which harbors the fused gene construct at its genome.

To examine early neurogenesis, extensive attempts have been made using zebrafish mutants in which the distribution of HuC mRNA is altered. In this connection, RNA in situ hybridization is used to screen the embryos for changes in the distribution of HuC-expressing cells. However, this in situ hybridization is disadvantageous in that it is impossible to examine a large number of live embryo mutants not only because embryos are killed by chemicals during the observation of development events, but because the experiment procedure is complicated.

According to the present invention, an embryological method by which changes in the early pattern of neurons can be visibly detected rapidly from live embryos is provided, thereby overcoming the limitation of the conventional RNA in situ hybridization. To this end, the isolated HuC promoter was used to create a zebrafish transformant which expresses GFP (green fluorescence protein) in a neuron-specific pattern.

In detail, a fused gene construct in which a GFP gene was located downstream of the HuC promoter (HuCP-GFP) was microinjected into one-cell stage zebrafish embryos. After two days of growth, embryos which showed neuron-specific expression of GFP were selected under a fluorescence microscope and raised to maturity. The recombinant plasmid in which a GFP gene was inserted downstream of the HuC promoter, named pHuC10GFP, was deposited with the Korean Collection for Type Culture of Korea Research Institute of Bioscience and Biotechnology (KRIBB) under the deposition No. KCTC 0802BP on Jun. 9, 2000. Further, the selected sperm which expresses GFP specifically in neurons was deposited with KRIBB under the deposition No. KCTC 0844BP on Jul. 27, 2000.

50 male and female zebrafish adults that had been grown from the embryos for three months, were crossed with wild-type male and female adults, and the progenies were tested for germline transmission of HuCP-GFP under the fluorescence microscope. One male adult which had shown GFP expression at an embryo stage was selected as a first-generation transgenic HuCP-GFP founder. When the selected transgenic founder male fish was crossed with a wild-type female zebrafish, the frequency at which the HUCP-GFP gene was inherited to the F₁ progeny from the first-generation transgenic founder by germline transmission was measured to be 12%. Upon reaching sexual maturity, male and female F₁ heterozygous transgenic zebrafishes (HuCP-GFP^{+/-}) were crossed with each other and approximately 25% of the F₂ embryos were identified as homozygous HUCP-GFP transgenics (HuCP-GFP^{+/+}) based on the level of GFP expression.

The expression level of GFP in the homozygous transgenic zebrafish was approximately two-fold higher than that in the heterozygous line, and neuron-specific GFP expression in the brain and spinal cord could be easily visualized (FIG. 7). The distribution of neurons in live zebrafish embryos can be visualized using confocal laser microscopy.

GFP transcription in the transgenic zebrafish embryos was detected by in situ hybridization using an antisense GFP RNA probe, at 11 hpf (hours post fertilization), which was close to the time point at which endogenous HuC transcripts were first seen in the wild-type zebrafish embryos. In all cases, GFP gene expression was found in the same region near the neural plate. This observation indicates that the neuron-specific expression of GFP in the transgenic zebrafish embryos follows the same pattern in terms of space and time as in the HUC transcripts of wild-type zebrafish embryos. Therefore, it was demonstrated that the HuC promoter isolated in the present invention is not only identified to comprise the complete regulatory region for the HuC gene which directs neuron-specific expression, but the expression of a GFP gene in the transgenic zebrafish is neuron-specific and shows the same pattern as the HuC gene of wild-type zebrafish.

In a further aspect of the present invention, there is provided a method for making the transgenic animal. The method can be broken down into the following five steps:

1) Preparing a fused gene construct in which a HuC promoter responsible for neuron-specific expression in zebrafish is ligated to a fluorescence protein gene.

2) Microinjecting the fused gene construct into embryos.

3) Selecting embryos showing neuron-specific expression of GFP.

4) Crossing adults of the selected embryos with wild-type adults to produce F₁ heterozygous transgenic progeny.

5) Self-crossing the F₁ heterozygous transgenic progeny with each other to produce F₂ homozygous transgenics.

In the step 1), the fluorescence protein gene may be selected from the group consisting of genes coding for GFP, luciferase and β-galactosidase. In a preferred embodiment, a recombinant plasmid for stable expression of GFP in neurons is constructed which contains the 5′-flanking region, exon-1, a part of exon-2 and the intervening intron-1 of HuC, and a GFP-encoding base sequence. This HuCP-GFP fused gene construct, named pHuC10GFP, was deposited with the Korean Collection for Type Culture of Korea Research Institute of Bioscience and Biotechnology (KRIBB) under the deposition No. KCTC 0802BP on Jun. 9, 2000.

In still a further aspect of the present invention, there is provided a method for visibly screening mutants whose nervous system is altered, with ease.

Large-scale mutagenesis screening processes have already identified a number of mutants in which the early pattern of neurons is altered. By taking advantage of the transgenic zebrafish of the present invention, living mutants in which the early pattern of neurons is altered can be visibly selected within a short period of time. The success in screening such mutants reflects not only the value of HuC as an early neuronal marker, but also that its promoter and the transgenic zebrafish created by using it are useful as a tool in the study on neurogenesis in vertebrates.

The method for screening neurogenesis mutants according to the present invention comprises the steps of:

1) crossing a homozygous zebrafish which harbors a HuCP-GFP fused gene construct in its genome with an unknown heterozygous neurogenesis mutant to produce F₁ progeny;

2) back-crossing F₁ progeny with the unknown heterozygous neurogenesis mutant to obtain homozygous neurogenesis mutants; and

3) comparing the GFP fluorescence between the homozygous neurogenesis mutant embryos and the F₁ progeny embryos.

To illustrate the usefulness of the screening method, the HuCP-GFP gene was introduced into mib (mind bomb) mutant zebrafish (Schier et al., 1996) which is characterized by a neurogenic phenotype with supernumerary early differentiating neurons and a deficiency in late differentiating neurons. In one preferred embodiment, homozygous HUCP-GFP transgenic zebrafish (HuCP-GFP^{+/+}) were crossed with heterozygous mib carriers (mib^{+/-}). Upon reaching sexual maturity, the resulting F₁ progeny (HuCP-GFP^{+/-}/mib^{+/-}) were back-crossed with the heterozygous mib mutant (mib^{+/-}) to obtain HuCP-GFP^{+/-}/mib^{-/-} mutant embryos. Making neuronal hyperplasia evident in HuCP-GFP^{+/-}/mib^{-/-} transgenic embryos, much more intense GFP fluorescence was observed in those transgenic embryos under a fluorescence microscope, compared to HuCP-GFP^{+/-} embryos. These results reflect how the screening method using the HuCP-GFP transgenic zebrafish could be used for isolating and analyzing neurogenesis mutants in living zebrafish with ease.

### EXAMPLES

A better understanding of the present invention may be obtained in light of the following examples which are set forth to illustrate, but are not to be construed to limit the present invention.

### Example 1

### Early Neuronal Expression of HuC in Zebrafish Embryo

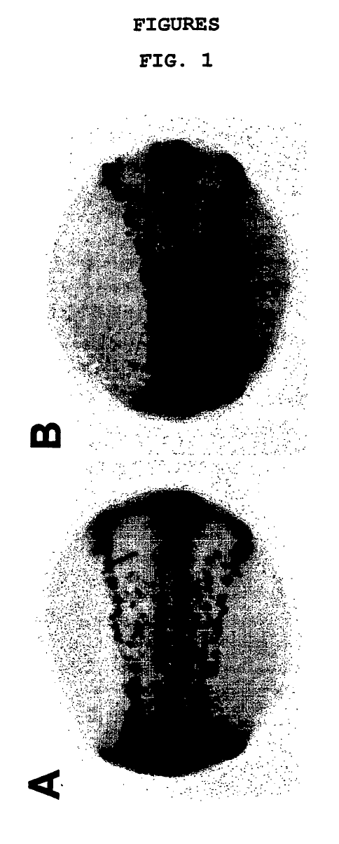

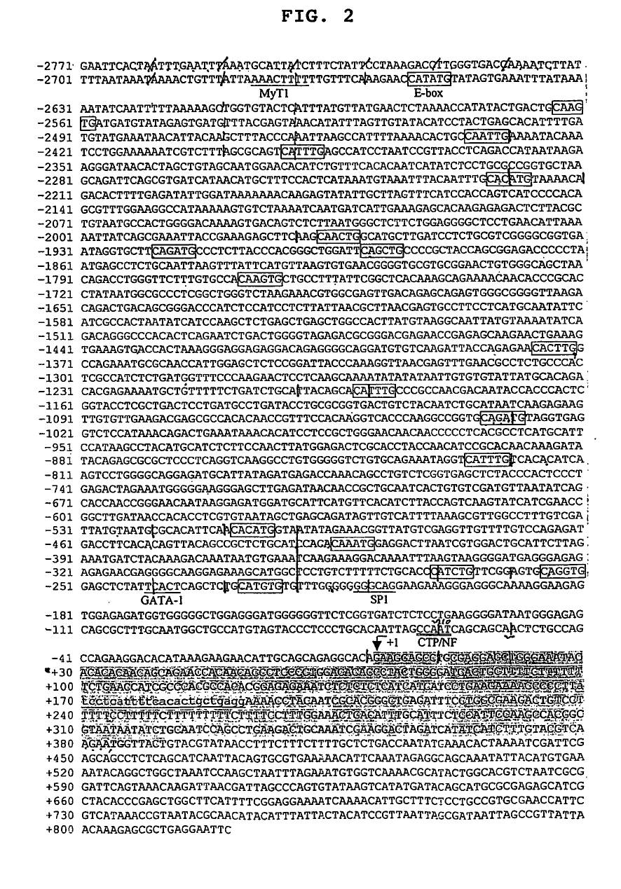

In a previous study, HuC was revealed to be a useful marker for neurons in zebrafish based on the fact that it is expressed in nascent primary neurons soon after gastrulation (Kim et al., 1996; Park et al., 2000). In this example, to provide additional evidence that HuC-positive cells are early neurons, the expression of HuC was compared with that of DeltaB, which has recently also been disclosed to be expressed in nascent neurons by recent studies (Haddon et al). With reference to FIG. 1, there are fluorescence photographs taken of dorsal parts of embryos, showing the comparison of HuC and DeltaB mRNA expression in the neural plate at the 3-somite stage. As shown at the sites of ps (primary sensory neuron), pin (primary intermediate neuron) and pmn (primary motor neuron) of the photographs, the expression of HuC (A) in three longitudinal columns within the neural plate is very similar to that of DeltaB (B) at the 3-somite stage.

### Example 2

### Isolation and Characterization of 5′-Flanking Region Containing Promoter for HuC Genomic DNA

In order to isolate the zebrafish HuC promoter region, a zebrafish genomic library was screened through hybridization using a radiolabeled probe derived from the 5′-UTR of zebrafish HuC cDNA (Kim et al., 1996). First, a zebrafish genomic DNA library (Clontech) was screened with [α-³²P]dCTP-labeled CDNA fragments containing the 5′-UTR of zebrafish HuC cDNA. A number of positive clones were identified by plaque hybridization. Of them, two clones containing a 15-kb NotI (clone 4) and a 18-kb NotI (clone 8) genomic DNA insert, respectively, were purified to single phage plaques. Preliminary restriction analysis and partial nucleotide sequencing resulted in the finding that a 7-kb NcoI DNA fragment of the 15-kb NotI genomic insert contained a 5-kb sequence upstream of the translation start codon ATG after the subcloning of the NcoI fragment into plasmid pGEM7(+) (Promega). To narrow the putative promoter region to a more defined one, an internal EcoRI fragment containing a 3.2-kb upstream sequence from the translation start codon ATG was isolated, followed by analyzing its complete nucleotide sequence by the dideoxynucleotide chain termination method (Sanger et al., 1977).

The transcription start site in the 5′-UTR of HuC cDNA, which was analyzed to have Sequence No. 1, was determined by primer extension using an antisense oligonucleotide derived from the 5′-UTR sequence.

Using T4 polynucleotide kinase (Promega), an oligonucleotide primer of Sequence No. 2 derived form the exon-1 of the zebrafish HuC gene was end-labeled with [γ-³²P]ATP (Amersham) to 10⁸ cpm/μg. 60 μg of total RNA isolated from each of 24-hpf zebrafish embryos and yeast tRNA were hybridized with the isotope-labeled primer (5×10⁵ cpm) at 30° C. After 18 hours of incubation, the reactions were precipitated by ethanol and resuspended in 20 μl of a reverse-transcriptase reaction mixture (50 mM Tris-Cl, 6 mM MgCl₂, 40 mM KCl, 10 mM dithiothreitol, pH 8.5). An AMV reverse transcriptase (Boehringer Mannheim) was added at an amount of 200 units to the reactions which were then incubated at 42° C. for 1 hour. After being precipitated in ethanol, the cDNA products were electrophoresed on 6% polyacrylamide gel containing 8 M urea. To map the nucleotide position for the transcription start site, a separate DNA sequencing reaction using a 3.6-kb EcoRI fragment of zebrafish HuC genomic DNA with the same oligonucleotide primer was performed and subjected to electrophoresis.

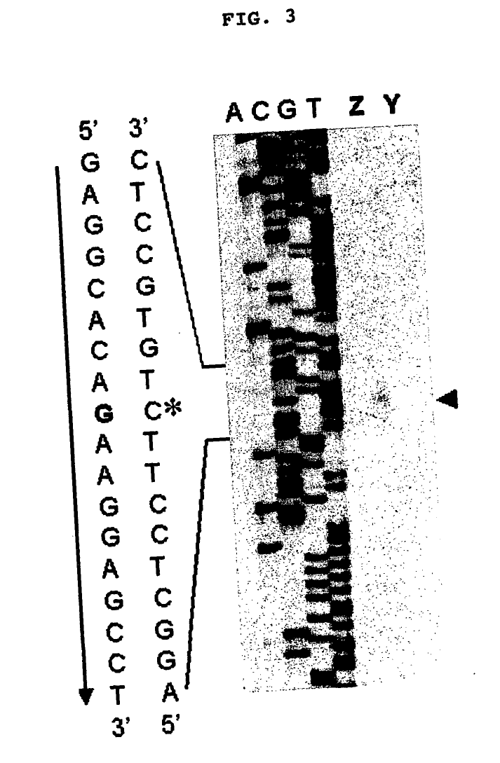

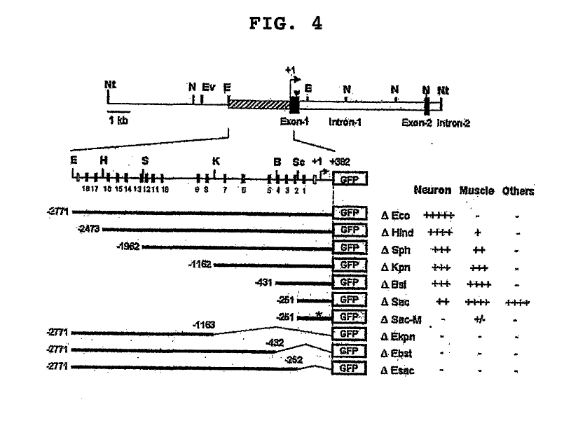

With reference to FIG. 3, there is shown an autoradiograph in which the transcription initiation site of the HuC gene is determined by primer extension. The Z lane is for the 24 hpf zebrafish embryos (Z) while the Y lane is for the yeast tRNA. An extended cDNA band from zebrafish RNA is indicated by the arrow and the corresponding nucleotide G is marked by an asterisk. As shown in this autoradiograph, a single cDNA band was extended on a template mRNA derived from 24-hpf zebrafish embryos. Using this cDNA, the nucleotide position of transcription initiation site was mapped within the genomic DNA and referred to as +1, and all subsequent nucleotide positions were numbered relative to this location, as shown in FIG. 2. The transcription initiation site mapped at G is consistent with the report that RNA polymerase II prefers to start at purines (Baker and Ziff, 1981).

To analyze the zebrafish HuC promoter, an examination was made of the GFP expression patterns in the neuron, muscle and other tissues of embryos by use of various deletion constructs. With reference to FIG. 4, there are shown structures of the zebrafish deletion constructs, along with their transient expression patterns. As seen in the schematic diagram of FIG. 4, a 3.6-kb EcoRI fragment of zebrafish HuC genomic DNA was identified to consist of 2,771 bp of the 5′-upstream sequence, 391 bp of exon-1 (382-bp 5′-UTR followed by a 9-bp coding sequence), and 429 bp of a part of intron-1 on the basis of the transcription initiation site and a previously reported HuC cDNA sequence. Analysis of the nucleotide sequence for the region immediately upstream of the transcription start site revealed the presence of one CCAAT box (−64/−60), one GATA-1 (−242/−238), and one SP1 (−213/−208) site, suggesting the possibility that the core promoter HuC is located around this region. However, there was no obvious TATA box near the region 30-bp upstream of the transcription initiation site. The most striking feature of the 5′-flanking sequence of the HuC gene is the presence of as many as 18 E-boxes as shown in FIG. 2, which indicates an important role for E-box-binding bHLH transcription factors in the neuron-specific regulation of HuC gene expression (Murre et al., 1994). This agreed with the previously suggested role of bHLH transcription factors such as ngn1 in the determination of neuronal fate. Furthermore, one putative MyT1 binding site, which has also been reported to be essential for neuronal differentiation, was identified at the nucleotide position −2687/−2680 as shown in FIG. 2.

### Example 3

### Identification of 5′-Flanking Region for Neuron-Specific Expression of HuC Gene

An examination was made to determine the size of the 5′-upstream sequence, containing the putative HuC promoter region, in the 3.6-kb EcoRI fragment, which is sufficient to restrict the expression of the GFP reporter gene to neurons.

First, a 3.2-kb (−2771/+382) genomic DNA fragment amplified by PCR from a template of the 3.6-kb EcoRI genomic DNA fragment, was fused with the GFP-encoding sequence of the plasmid pEGFP-1 (Clontech) at the EcoRI/SmaI site to construct a HuCP-GFP gene, designated ΔEco. The PCR was performed using pfu Turbo DNA polymerase (Stratagene).

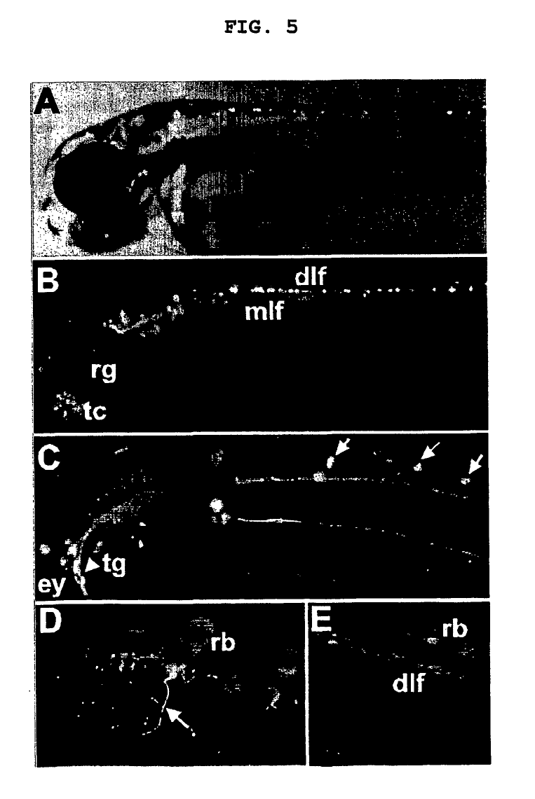

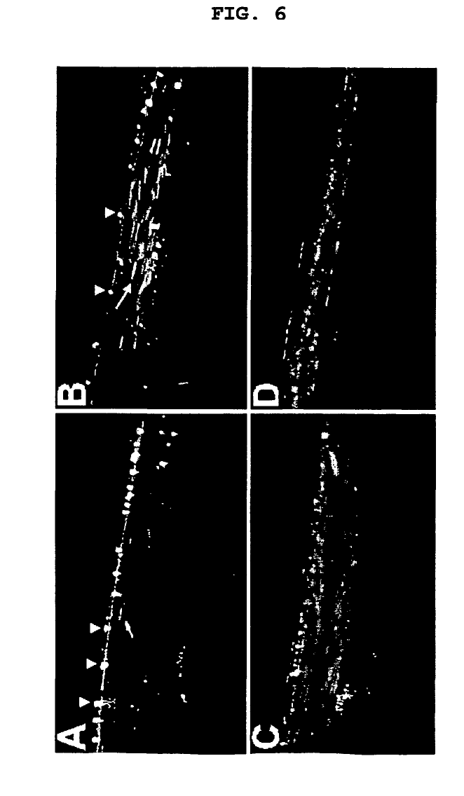

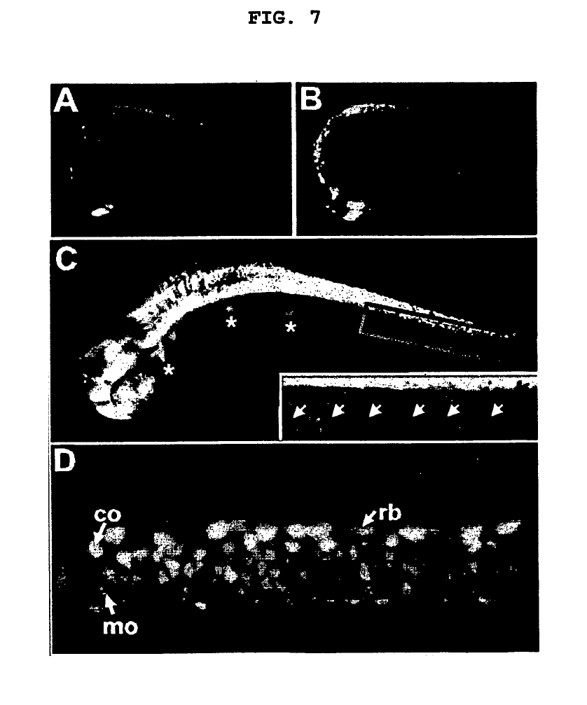

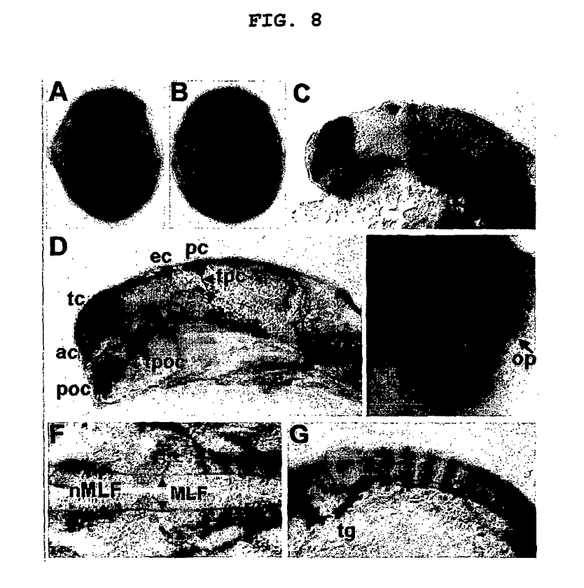

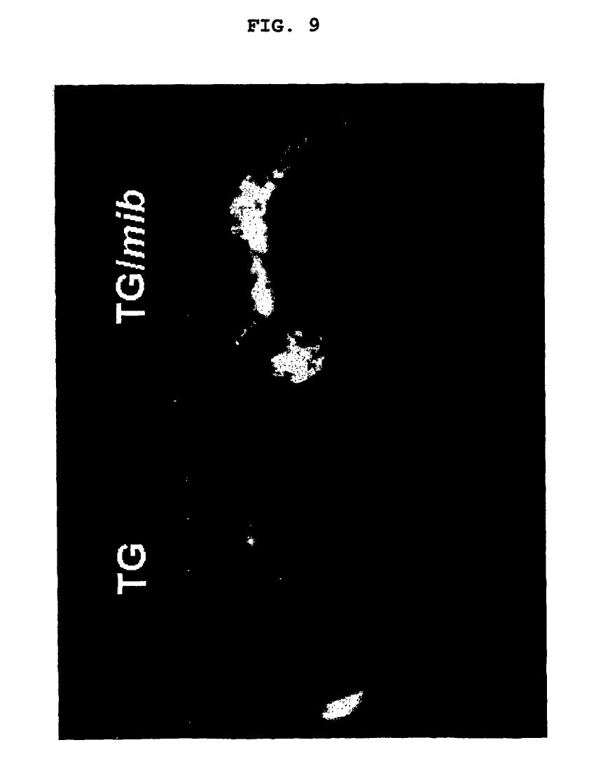

The ΔEco DNA construct was microinjected into zebrafish embryos at the one-cell stage and its control in gene expression was analyzed by observing the GFP expression in the embryos under a fluorescence microscope. At 48 hpf, embryos microinjected with ΔEco were found to express GFP in all regions of the nervous system. The results are given in FIG. 5. As shown in the fluorescence photographs of FIG. 5, the telencephalic cluster, the retinal ganglion neuron, the trigeminal ganglion neuron, medial longitudinal fasciculus and dorsal longitudinal fasciculus are the sites in which GFP was most easily observed. Also, the peripheral projections of Rohon-Beard neurons as well as their central projections that terminate in the hindbrain could be easily identified by the strong fluorescence of GFP. Additionally, the major axonal tracts that make up the early axonal scaffold in the brain were visualized by the strong GFP expression in axons.

Furthermore, the neuronal specificity of the GFP expression driven by the ΔEco was identified again in whole mounts with an anti-GFP polyclonal antibody, indicating that the 5′-flanking promoter region in the ΔEco construct contains all regulatory elements necessary to restrict HuC gene expression to the neurons.

### Example 4

### Functional Analysis of HuC Promoter in Zebrafish Embryos

For the identification of regulatory regions necessary to maintain HuC gene expression exclusively in the neurons, serial deletions of the 5′-flanking region in the ΔEco construct were generated from both 5′- and 3′-ends, as shown in FIG. 4.

To this end, first, the ΔEco construct was cleaved with EcoRI/HindIII, EcoRI/SphI, EcoRI/KpnI, EcoRI/BstXI and EcoRI/SacI. Thereafter, larger DNA fragments from each of the restriction reactions were isolated and self-ligated to yield ΔHind (−2473 to +382 bp), ΔSph (−1962 to +382 bp), ΔKpn (−1161 to +382), ΔBst (−431 to +382) and ΔSac (−251 to +382) constructs. Separately, the ΔEco construct was also digested with EcoRI/KpnI, EcoRI/BstXI, and EcoRI/SacI, and the smaller DNA fragments were inserted into the compatible sites in plasmid pEGFP-1. When appropriate restriction sites were not available, 3′-ends were blunted with klenow enzyme and inserted into the EcoRI/SmaI site. The CCAAT-box sequence in the ΔSac construct was mutated to CCCAT by site-directed mutagenesis using a site-directed mutagenesis kit (Stratagene) with the oligonucleotide primer of Sequence No. 3 to give a ΔSac-M construct.

Changes in GFP expression resulted from the deletions were identified by examining GFP expression at 48 hpf in embryos injected with specific deletion constructs at the one-cell stage. The results are shown in FIG. 6.

When embryos were injected with the ΔHind construct (−2473/+382), the expression of GFP in neurons was similar to that with the ΔEco construct. The GFP expression, however, was also observed in muscle cells (FIG. 6A). This result suggests the role of a putative MyT1 binding site (−2687/−2680) and/or two E-box sequences (17^{th} at −2565/−2560 and 18^{th} at −2665/−2650) (FIGS. 2 and 4) in the suppression of HuC expression in muscle cells. Since MyT1 is not expressed in muscle cells, it is more likely that loss of the E boxes in this deletion mutant leads to the more promiscuous expression of GFP.

When the 5′-flanking region of the HuC promoter was progressively deleted toward the 3′-end, GFP expression was increased only in muscle cells without concomitant loss of GFP expression in neurons. That is, the expression intensity of GFP in muscle cells increased in the order of the microinjection with constructs ΔHind (−2473/+382), ΔSph (−1962/+382), ΔKpn (−1162/+382), and ΔBst (−431/+382). Finally, GFP expression in muscle cells driven by the ΔBst construct increased to the extent of overwhelming its expression in neuronal cells as shown in FIG. 6B. These results indicate that the 12 E-box sequences (5-16) play a more important role in the suppression of HuC expression in muscle cells than in the neuron-specific expression of HuC.

In contrast, the ΔSac (−251/+382) construct drives ubiquitous expression of GFP in all tissues, including skin and notochord and neurons, of most embryos, giving the suggestion that the proximal three E-boxes present in the ΔBst construct are indispensable for the maintenance of neuron-specific expression of HuC as shown in FIGS. 6C and 6D.

To test the function of the putative CCAAT-box, a point mutation was introduced into the ΔSac construct to change the first A to C. The resulting ΔSac-M construct was found to almost completely lose its promoter activity, as illustrated in FIG. 4. Therefore, a 5′-flanking region spanning 251 bp in the ΔSac construct was proved to represent a core promoter for the HuC gene.

This result, that is, the localization of a core promoter region within the ΔSac construct, was confirmed by testing GFP expression with ΔEbst (−2771/−431), ΔEkpn (−2771/−1162), and ΔEsac (−2771/−251) constructs, which all lack the 251-bp 5′-flanking region of the ΔSac construct. Embryos injected with ΔEbst (−2771/−431), ΔEkpn (−2771/−1162) and ΔEsac (−2771/−251) constructs did not show any significant GFP expression, supporting the role of the 251-bp 5′-flanking sequence as the core promoter for the zebrafish HuC gene. In addition, these results indicate that 17 E-box sequences and one MyT1 binding site, along with the proximal core promoter region, orchestrate the neuron-specific expression of HuC.

### Example 5

### Creation of Transgenic Zebrafish Capable of Neuron-Specific Expression of GFP

5-1: Construction of Fused Gene

For the stable expression of GFP in neurons, a fused gene construct (hereinafter referred to as “HuC promoter-GFP’ or “HuCP-GFP”) was prepared consisting of exon-1, intron-1, a part of exon-2, and a GFP-encoding sequence.

Using the clone #4 which harbors the 15-kb genomic DNA fragment prepared in Example 2, the HUCP-GFP fused gene was constructed as in the following consecutive recombination processes. To begin with, plasmid pEGFP-C1 DNA was double-digested with Eco47III/XhoI, followed by inserting the resulting 0.75-kb GFP DNA digest into the StuI/XhoI site of the plasmid vector CS2A(−) which was previously derived from the self-ligation of the large fragment remaining after the removal of the CMV promoter when plasmid CS2(−) was digested with SalI/HindIII. The resulting recombinant plasmid CS2A(−)-GFP was further cleaved with NcoI, after which the HuC promoter containing, 10.5-kb NcoI digest from the 15-kb HuC genomic DNA of clone #4, which contains 4.6 kb of the 5′-flanking region, 391 bp of exon-1, 5.5 kb of intron-1, and 15 bp of exon, was inserted into the NcoI site of the recombinant plasmid CS2A(−)-GFP so that the GFP gene was regulated under the HuC promoter. In addition, this insertion brought about the effect of newly replacing the translation initiation codon ATG of the GFP gene which was lost upon the excision of the GFP gene from the plasmid. Finally, the resulting recombinant plasmid CS2A(−) containing the 10.5-kb HuC gene and the 0.75-kb GFP gene was further digested by EcoRV/BamHI to remove the 0.5-kb EcoRV/NcoI DNA fragment at the most 5′-upstream sequence of the 5′-flanking sequence in the 4.6-kb HuC genomic DNA, and then self-ligated to construct a HuCP-GFP fused gene expression plasmid. This resulting recombinant expression vector was linearized by a single-cut restriction enzyme ScaI and the linearized forms of DNA were microinjected into one-cell stage embryos. The recombinant plasmid pHuC10GFP, which contains the HuCP-GFP fused gene construct, was deposited with the Korean Collection for Type Culture of Korea Research Institute of Bioscience and Biotechnology (KRIBB) under the deposition No. KCTC 0802BP on Jun. 9, 2000.

5-2: Preparation of Zebrafish Embryos

Zebrafish were raised at 28° C. with cycles of 14 hours in the daylight and 10 hours in the dark. Until the time of crossing, male and female were grown in separate tanks. Upon mating, beads were laid sufficiently to completely cover the bottom of the incubation bath lest the adults eat the eggs. Under a light, the fertilized eggs were harvested at appropriated intervals of 1-2 hours with the aid of a tube. After being raised for 2-4 days in incubation water containing 60 μg/ml of sea salts (Sigma), the embryos microinjected with the recombinant plasmids and control embryos were transferred to a common water bath for growth. Zebrafish were maintained with care according to a well-known process (Westerfield, 1995).

5-3: Preparation of Embryos Microinjected with HuCP-GFP Fused Gene Construct

The recombinant plasmid CS2A(−) DNA containing the HuCP-GFP fused gene construct was microinjected into 500 one-cell stage zebrafish embryos. 48 hours after microinjection, embryos which transiently expressed GFP in neurons were identified by fluorescence microscopy and raised to sexual maturity.

For use in microinjection, the fused gene construct was prepared using EndoFree Plasmid kit (Qiagen). In this regard, the HuCP-GFP fused gene expression plasmid was linearized with an appropriate restriction enzyme and isolated through the extraction in phenol-chloroform and the precipitation by ethanol. Zebrafish embryos were stored in plastic vessels with a diameter of 10 cm and microinjected with DNA in advance of the first cleavage under a dissecting microscope. For microinjection, DNA concentration was adjusted to 100 μg/ml in 0.1 M KCl solution (Stuart et al., 1990) containing 0.5% phenol red, and the solution with such a DNA concentration was injected into the one-cell stage embryos at an amount of 100-200 pl per embryo prior to the first cleavage.

Adults were crossed with wild-type fish and progeny were tested for germline transmission of HuCP-GFP under a fluorescence microscope. One male adult capable of germline transmission was identified as a transgenic HuCP-GFP founder fish and back-crossed with wild-type female fish. As a consequence, twelve percent of the F₁ progeny was found to inherit the HuCP-GFP gene by germline transmission from the founder. Upon reaching sexual maturity, male and female F₁ heterozygous transgenic zebrafish were crossed with each other to yield F₂ progeny, approximately a quarter of which were identified as homozygous transgenic zebrafish (HuCP-GFP^{+/+}). The selected sperm of the homozygous transgenic zebrafish microinjected with the plasmid pHuC10GFP capable of directing the neuron-specific expression of GFP were deposited with KRIBB under the deposition No. KCTC 0844BP on Jul. 27, 2000.

### Example 6

### Identification of Regulation Pattern of HuC Promoter in Transgenic Zebrafish Neuron

An examination was made of the HuC promoter-driven GFP expression in neurons of the HuCP-GFP transgenic zebrafish prepared in Example 4. The expression level of GFP in the homozygous transgenic zebrafish was approximately two-fold higher than that in the heterozygous line, and neuron-specific GFP expression in the brain and spinal cord could be easily visualized, as shown in FIG. 7. Additionally, the HuCP-GFP transgenics made it possible to visualize the detailed distribution of neurons in live zebrafish embryos under a confocal laser microscope. In detail, at approximately 24 hpf, clear GFP expression could be identified in primary commissural neurons, Rohon-Beard neurons and motorneurons of the spinal cord by their bright fluorescence, showing in detail the precise positions of neurons, according to type (FIG. 7D).

In order to determine whether the spatial and temporal GFP expression in the HuCP-GFP transgenic zebrafish is similar to the spatial and temporal expression of HuC mRNA in wild-type zebrafish embryos, RNA in situ hybridization was conducted as follows. First, an antisense digoxigenin-labeled RNA probe for the 3′-UTR of zebrafish HuC cDNA was produced using a DIG-RNA labeling kit (Boehringer Manheim), followed by performing hybridization and detection with an antidigoxigenin antibody coupled to alkaline phosphatase according to the instruction of Jowett and Lettice (Jowett and Lettice, 1994).

By RNA in situ hybridization using the antisense GFP RNA probe, the GFP transcription in the transgenic zebrafish embryos was detected at 11 hpf, which was close to the time point at which endogenous HuC transcripts were first seen in the wild-type zebrafish embryos (FIGS. 8A and 8B).

For the examination of the neuronal specificity of GFP expression in the HuCP-GFP transgenic lines, GFP-positive cells in the transgenic zebrafish embryos were visualized by a whole-mount immunostaining method using an anti-GFP polyclonal antibody.

In more detail, dechorionated embryos were fixed in BT buffer (0.1 M CaCl₂, 4% sucrose in 0.1 M NaPO₄, pH 7.4) containing 4% paraformaldehyde for 12 hours at 4° C., and then rinsed in PBST (1×PBS, 0.1% Triton X-100, pH 7.4). After being frozen in acetone at −20° C. for 7 min, the embryos were washed three times with PBST, and immersed for 1 hour in a PBS-DT blocking solution. (1×pBST, 1% BSA, 1% DMSO, 0.1% Triton X-100, 2% goat serum). Then, the embryos were reacted with 1:1000 diluted anti-GFP polyclonal antibody (Clonetech) for 4 hours at room temperature, washed 10 times for 2 hours with PBS-DT, and incubated with 1:500 diluted biotinylated goat anti-rabbit antibody (Vector) at 4° C. overnight. The embryos were washed for 6 hours in PBS-DT, incubated for 2 hours at room temperature in Vectastain Elite ABC reagent (Vector), washed five times in PBS-DT, and washed three times in 0.1 M NaPO₄. Afterwards, the embryos were incubated with 1 ml of DAB solution (1% DMSO, 0.5 mg/ml diaminobenzidine, 0.0003% H₂O₂ in 0.05 M NaPO₄, pH 7.4) at room temperature. When a color change was observed while monitoring the embryos for 5 to 10 min under a dissecting microscope, the chromogenic reaction was stopped by the addition of a 0.1 M NaPO₄ solution (pH 7.4).

Patterns of whole-mount in situ hybridization patterns and immunostaining were observed using a Zeiss Axiocop microscope. Embryos and adult fish were anesthetized using tricaine (Sigma) according to the instruction of Westerfield (1995), and examined through an FITC filter on a Zeiss Axioskop fluorescence microscope. Laser confocal microscopic images were obtained using Leica DM/R-TCS laser scanning microscope equipped with an FITC filter.

In 24-hpf transgenic zebrafish embryos, various neurons, including GFP expression in telencephalic cluster, anterior commissure, epiphyseal cluster, posterior commissure, tract of posterior commissure, postoptic commissure, tract of the postoptic commissure, olfactory placodes, nuclei of medial longitudinal fasciculus, medial longitudinal fasciculus, trigeminal ganglion, seven rhombomeres in the hindbrain, were recognized by the anti-GFP antibody as shown in FIG. 8. Further, early motorneurons, Rohon-beard neurons and interneurons of the spinal cord were also detected by the anti-GFP antibody in the same GFP expression pattern as that observed under the laser confocal microscope. These results indicate that the GFP RNA expression in the transgenic line is temporally and spatially similar to that of HuC mRNA transcripts in the wild-type zebrafish.

### Example 7

### Characterization of Neurogenesis Mutant Using HuCP-GFP Transgenic Zebrafish

With the aim of identifying the usefulness of HuCP-GFP transgenic zebrafish as a useful tool for characterizing neurogenesis mutants, the HUCP-GFP gene was introduced into the mib mutant zebrafish (Schier et al., 1996). The mib mutant is known as a neurogenic phenotype of neural hyperplasia, in which supernumerary early differentiating neurons exist.

To begin with, homozygous HuCP-GFP zebrafish (HuCP-GFP^{+/+}) were crossed with heterozygous mib carriers (mib+/−). Upon reaching sexual maturity, the resulting F₁ progeny (HuCP-GFP^{+/-}/mib^{+/-}) were back-crossed with the heterozygous min carriers (mib^{+/-}) to yield heterozygous mutant embryos (HuCP-GFP^{+/-}/mib^{+/}⁻). Not only much more intense GFP fluorescence, but also more extensive GFP expression regions were detected in the F₂ heterozygous mutant embryos (HuCP-GFP^{+/-}/mib^{+/-}) than in the heterozygous transgenic embryos (HuCP-GFP+/−), demonstrating that neuronal hyperplasia occurs in HuCP-GFP^{+/-}/mib^{+/-} transgenic embryos, as shown in FIG. 9. Therefore, the HuCP-GFP transgenic zebrafish of the present invention can be useful for isolating and analyzing neurogenesis mutants in zebrafish.

### Industrial Applicability

As described hereinbefore, the HuC promoter, whose expression is a useful early marker for neurons in zebrafish, is isolated and characterized for base sequence, regulatory element, and neuron-differentiating mechanism, in accordance with the present invention. Also, the present invention provides a transgenic zebrafish line that expresses GFP specifically in neurons. In addition, the HuC promoter of the present invention can be used in the study of the regulatory mechanism responsible for the differentiation of the nervous system. Taken together, these results indicate that the HuCP-GFP transgenic zebrafish of the present invention enable the direct identification of neurogenesis and axonogenesis, as well as being a valuable tool for isolating and analyzing neurogenesis mutants in live zebrafish with ease.

The present invention has been described in an illustrative manner, and it is to be understood that the terminology used is intended to be in the nature of description rather than of limitation. Many modifications and variations of the present invention are possible in light of the above teachings. Therefore, it is to be understood that within the scope of the appended claims, the invention may be practiced otherwise than as specifically described.

What is claimed is:

1. A HuC promoter with its 5′-flanking region, capable of driving gene expression specifically in neurons.

2. The HuC promoter with its 5′-flanking region as set forth in claim 1, wherein the HuC promoter has the base sequence listed in Sequence No. 1.

3. A recombinant plasmid pHuC10GFP, deposited under the deposition No. KCTC 0820BP, in which a green fluorescence protein (GFP) gene is ligated to the HuC promoter of claim 1.

4. A sperm of a homozygous transgenic zebrafish, deposited under deposition No. KCTC 0844BP, containing the recombinant plasmid of claim 3.

5. A zebrafish, which harbor the recombinant plasmid of claim 3 in their genome and show neuron-specific expression of GFP.

6. A method for generating a transgenic animal, comprising the steps of:
preparing a fused gene construct in which a HuC promoter responsible for neuron-specific expression in zebrafish is ligated to a fluorescence protein gene;
microinjecting the fused gene construct into embryos;
selecting embryos showing neuron-specific expression of GFP;
crossing adults of the selected embryos with wild-type adults to produce F₁ heterozygous transgenic progeny; and
self-crossing the F₁ heterozygous transgenic progeny with each other to produce F₂ homozygous transgenics.

7. The method as set forth in claim 6, wherein said fluorescence protein gene is selected from genes coding for GFP, luciferase and β-galactosidase.

8. The method as set forth in claim 6, wherein said fused gene construct contains the 5′-flanking region, exon-1, a part of exon-2 and the intervening intron-1 of HuC, and a GFP-encoding base sequence.

9. The method as set forth in claim 6, wherein said transgenic animal is zebrafish.

10. A method for screening neurogenesis mutants in zebrafish, in which the transgenic zebrafish of claim 5 is utilized.

11. The method as set forth in claim 10, in which the method comprises the steps of:
crossing a homozygous zebrafish which harbors a HuCP-GFP fused gene construct in its genome, with an unknown heterozygous neurogenesis mutant to produce F₁ progeny;
back-crossing F₁ progeny with the unknown heterozygous neurogenesis mutant to obtain homozygous neurogenesis mutants; and
comparing the GFP fluorescence between the homozygous neurogenesis mutant embryos and the F₁ progeny embryos.

12. A method for analyzing alterations in the nervous system, in which the transgenic zebrafish of claim 5 is utilized.
